(19) United States
(12) Patent Application Publication
(10) Pub. No.: US 2026/0235595 A1
(43) Pub. Date: Aug. 13, 2026

(54) METHOD FOR DETERMINING THE CHARGE STATE OF PROTEINS

(71) Applicant: PAIA BIOTECH GMBH, Koeln (DE)

(72) Inventors: Kristina LECHNER, Koeln (DE); Christine WOSNITZA, Koeln (DE); Aris PERROU, Koeln (DE); Sebstian GIEHRING, Koeln (DE)

(21) Appl. No.: 19/154,250

(22) PCT Filed: Feb. 13, 2024
(86) PCT No.: PCT/EP2024/053570

§ 371 Date: Aug. 6, 2025

(30) Foreign Application Priority Data
Feb. 13, 2023 (EP) 23156423.8

(51) Int. Cl. G01N 33/543; G01N 21/64; G01N 33/68; G01N 33/84

(52) U.S. Cl. CPC G01N 33/54313; G01N 21/6428; G01N 33/68; G01N 33/84; G01N 2021/6441

(57) ABSTRACT

The invention relates to a method for analyzing charge variants of proteins using microtiter plates whose wells have at least partially different pH values and/or salt concentrations and using functionalized surfaces.

The application relates to methods for analyzing charge variants of proteins using microtiter plates whose wells have at least partially different pH values and/or salt concentrations and using functionalized surfaces.

The production of proteins and, in particular, the production and development of biopharmaceuticals, including virus particle-based therapeutics, is of great economic importance. However, the production of proteins and, in particular, biopharmaceuticals is very complex and time-consuming. Biopharmaceuticals are more heterogeneous in terms of their physical properties than chemically produced drugs, known as small molecule drugs.

Biopharmaceuticals, including virus particle-based therapeutics, are generally pharmaceutically useful and effective proteins, such as antibodies and virus particles.

During the production and handling of proteins, and in particular biopharmaceuticals, undesirable by-products can arise as a result of synthesis errors, degeneration or other reactions; which undesirable by-products must be identified and separated, as these often influence the biological efficacy of the protein.

The by-products are mostly incorrectly assembled, e.g., mispaired proteins or fragments of these proteins, or different charge variants of the target protein.

The different charge states of the charge variants of the biopharmaceutical, especially antibodies, Fc fusion proteins, antibody fragments, bi- and multispecific antibodies, and virus particle-based therapeutics, can lead to significant differences in physicochemical behavior, such as structure, stability, and binding affinity, and thus influence therapeutic efficacy.

In the production of proteins, especially biopharmaceuticals, it is therefore important to find cell lines and cultivation conditions that result in as few faulty assemblies and charge variants as possible. In this so-called upstream development often hundreds of samples are generated that must be tested for suitability in a short period of time.

The same applies to downstream development, in which the purification of proteins, especially biopharmaceuticals, is optimized.

Mispairing occurs, for example, when, after the expression of bi- or multispecific antibodies, the different amino acid chains which were produced are not assembled in the desired manner. Despite various approaches to optimize assembly and to direct and shift the assembly toward the desired target protein, this mispairing remains a major problem.

The primary goal in the development of processes for the production and purification of proteins, especially biopharmaceuticals, should therefore be to find conditions under which as few by-products as possible are produced. This must be determined experimentally and requires close monitoring of the experimental solution (e.g., cell culture) in order to be able to make changes to the experimental conditions in a timely manner.

Virus particles used in gene therapy, for example, can also have different charges. Loading the virus particles with nucleic acids (DNA or RNA) results in the virus particle surface having a stronger negative charge than virus particles that are not loaded or only partially loaded. Since the loading of nucleic acids onto the virus particles is essential for the therapeutic effect, it is very important to ensure that the virus particles are loaded during the production process of such virus particles. Virus particles that are not loaded with nucleic acids are non-functional by-products and must be removed.

Various methods are available to detect the presence or absence of nucleic acids in virus particles. These include PCR methods in combination with ELISA methods, cryo-electron microscopy, dynamic light scattering (DLS), analytical ultracentrifugation, or multi-step methods with various biosensors using biolayer interferometry. In addition, nucleic acids influence the charge of the virus capsid, enabling separation based on their charge in ion exchange chromatography.

New methods, tools, and devices are constantly being developed to simplify the manufacturing methods and handling of proteins, especially biopharmaceuticals, and thus make them more cost-effective.

The task of the present invention is to develop methods for rapidly detecting charge variants of a target protein, namely antibodies, Fc fusion proteins, antibody fragments, bi- and multispecific antibodies, and virus particle-based therapeutics, as well as incorrectly assembled (e.g., mismatched or fragmented) proteins. The latter is based on the fact that the target proteins and the proteins that are incorrectly assembled during their production very often have different charges and thus different isoelectric points.

Methods and apparatus for detecting and quantifying proteins based on their physical parameters are known.

A method frequently used for the characterization and purification of biological molecules is column chromatography. It can be performed both under normal pressure and under elevated pressure. The latter often requires expensive equipment such as HPLC or FPLC.

Another method is based on the use of biosensors, as described, for example, in U.S. Pat. No. 5,804,453 B.

A biosensor is a device that combines a biological component with a physical or chemical transducer to generate a measurable signal in response to a target analyte. Biosensors can use various physical measurement principles to detect the presence of target analytes.

Known examples include piezoelectric biosensors, optical detection methods based on interferometry or surface plasmon resonance, and calorimetric measurement methods.

Biosensors that use interferometric methods are described, for example, in U.S. Pat. No. 5,804,453 B. Biosensors that use piezoelectric methods are also known and are described, for example, in EP2017613A1.

Biosensors that use interferometric methods are particularly frequently used in combination with microtiter plates because the biosensors are easy to manufacture and, due to their shape, can be easily immersed in the wells of the microtiter plates.

The biosensors used in so-called biolayer interferometry have two reflective surfaces on which a phase shift is measured when biomolecules attach themselves to the second surface, which is further away from the light source.

Biosensor surfaces must be functionalized for the binding of the target analytes. The surfaces are often functionalized with amines to enable the further coupling of proteins or peptides via an amide bond, as described, for example, in WO 2008/033535.

Functionalization is achieved by bifunctional linkers, which react with the surface of the biosensor on the one hand and provide a functionality that enables the binding of the actual binding molecule (e.g., an antibody) on the other. For glass surfaces, these can be siloxanes, for example, a classic method for functionalizing glass surfaces. For polymer surfaces, copolymers made of polystyrene and divinylbenzene, for example, can be used, which provide reactive groups for further functionalization of the polymer surfaces. These coupling methods are also suitable for introducing groups for cation or anion exchange.

While column chromatography methods are used to detect and optionally to identify charge variants and incorrectly assembled proteins, the use of biosensors is not yet known.

The disadvantage of column chromatography methods is always that the samples must be pre-purified before analysis. This is important because otherwise the column material is very quickly irreversibly inactivated and because foreign proteins (unwanted proteins) cannot be distinguished from the target protein by the most commonly used UV absorption measurement at, for example, 280 nm. Regeneration of the column material between runs is also necessary and time-consuming.

First, the sample is adsorbed onto a so-called stationary phase (the column material, which can consist of various materials). By adding eluents (mobile phase(s)), i.e., solutions with different properties (salt content/pH value), the ionic interactions of the analytes with the stationary phase are weakened, the adsorbed molecules are desorbed and eluted.

In cation exchange chromatography (CEX), negatively charged column material is used in combination with a pH and/or salt gradient. If a pH gradient is used, the process begins with a low pH value. At this value, the biological molecules (e.g., proteins) in the sample are relatively strongly protonated and thus positively charged. This ensures that the molecules bind very well to the column material. As the pH value increases, the biological molecules are deprotonated, lose their positive charge, no longer bind to the stationary phase, and elute successively depending on their charge. Anion exchange chromatography (AEX) works in a similar way, but with positively charged column material.

Together with the pH gradient or instead of the pH gradient, a salt gradient can also be used to separate different molecules. This involves starting with low salt concentrations and increasing the salt concentration during the chromatographic separation. As a result, the salt (e.g., NaCl), especially the cations (e.g., Na+), increasingly binds to the negatively charged column material and displaces the positively charged biological molecules bound there, which then elute from the column material.

A pH gradient or salt gradient refers to a change in the pH value or hydrogen ion concentration and/or salt concentration over a specific spatial or temporal range.

Mixed-mode ion chromatography is also used for certain complex protein separations. Mixed-mode ion chromatography is a further development of ion exchange chromatography. It uses surfaces (e.g., in the form of particles) that have both positively and negatively charged groups. Another option is multi-mode ion exchange chromatography, in which surfaces (e.g., in the form of particles) are used that can enter into hydrophobic interactions and hydrogen bonds in addition to ionic interactions.

The aim of the present invention is to provide devices and methods for the direct, efficient, and rapid qualitative and quantitative determination of proteins, such as biopharmaceuticals, in particular antibodies, Fc fusion proteins, antibody fragments, bi- and multispecific antibodies, and virus particle-based therapeutics, without prior sample purification. The measurement should also be simple and preferably feasible using routine laboratory equipment.

The inventors have now found the methods, uses, and devices as protected by the present claims that can be used to characterize and separate proteins based on their charge differences. This enables users to quickly and easily identify by-products based on their charge state. The disadvantages of the aforementioned methods (lengthy purification processes prior to the actual analysis, etc.) are thus avoided.

The invention relates in particular to a method for analyzing charge variants of proteins using microtiter plates whose wells have at least partially different pH values and/or salt concentrations and using functionalized surfaces.

The invention is preferably used for analyzing charge variants of biopharmaceuticals, in particular therapeutically active proteins, such as antibodies and protein formats derived therefrom, such as Fc fusion proteins, antibody fragments, bi- and multi-specific antibodies, and virus particle-based therapeutics.

The functionalized surfaces are preferably ion exchange particles, in particular anion or cation exchangers, mixed-mode and multi-mode ion exchangers, or sensors such as biosensors whose measuring surface is designed to bind charged molecules and whose shape is such that they can be immersed in the wells of microtiter plates.

If marker molecules, such as mono- or polyclonal antibodies or fragments derived therefrom such as nanobodies or single-domain antibodies as well as peptides and RNA- or DNA-based aptamers are used in the method according to the invention, the biopharmaceutical, the target protein, has binding sites for the marker molecule or molecules. The marker molecules may also comprise dyes, such as fluorescent dyes. Such markers are referred to below as fluorescent markers.

The use of markers, in particular fluorescent markers in combination with the PAIA plate, has the advantage that other proteins which, due to their charge, can bind to the functionalized surfaces but are not bound by the marker molecule(s), are not detected. Such proteins include host cell proteins, as typically found in cell culture supernatants, or misassemblies in the production of bi- or multi-specific antibodies that no longer have a binding site for the marker molecule(s).

Marker molecules are molecules that can specifically bind to the target molecules. Examples of marker molecules are mono- or polyclonal antibodies, or fragments derived from these, such as nanobodies or single-domain antibodies, as well as peptides and RNA- or DNA-based aptamers.

Fluorescent markers are marker molecules to which a fluorescent dye is bound. The dyes can be covalently bound to the fluorescent markers or, for example, via the well-known streptavidin-biotin system, in which a fluorescently labeled streptavidin or other fluorescently labeled biotin-binding proteins such as avidin or neutravidin are coupled to a biotinylated marker molecule.

The skilled person can easily determine which fluorescent markers are particularly suitable for the method and use according to the invention. Examples of fluorescent markers that can be used according to the invention are Alexa 647-conjugated Affibody from Affibody AB or Nano-Secondary® alpaca anti-human IgG/anti-rabbit IgG, recombinant VHH, Alexa Fluor® 647 (Art. No. CTK0101-ChromoTek GmbH). Other fluorescent markers are commercially available, e.g. from Jackson Immunoresearch or Abcam. These are already coupled with a fluorescent dye and can be used directly. Alternatively, mono- or polyclonal antibodies can be purchased from these companies and coupled with various commercially available dyes, e.g., fluorescein, as well as dyes from Biotium, Atto-Tec, and ThermoFisher.

When selecting the fluorescent marker, care must be taken to ensure that the fluorescent marker does not bind directly to the functionalized surfaces under any of the assay conditions (pH value and salt). This can be easily determined by a skilled person, e.g., by examining mixtures of the surfaces with the fluorescent marker under a fluorescence microscope.

The method is primarily used for analysis, i.e., the identification and, if necessary, quantification of differently charged proteins (also referred to here as “charge variants of proteins”). Proteins include biopharmaceuticals (i.e., therapeutically active proteins) such as antibodies, Fc fusion proteins, antibody fragments, or bi- and multi-specific antibodies as well as virus particle-based therapeutics.

Charge variants may be the same protein (i.e., a protein with the same amino acid sequence) with multiple charge states. Examples include antibodies that are differently charged due to different glycosylation, amidation, or oxidation of amino acids.

Charge variants also include incorrectly assembled proteins. These have different amino acid sequences or different types of protein domains and, in some cases, a different number of protein domains, such as light and heavy chains in antibodies, and are therefore often differently charged and have different isoelectric points. Similarly, differently charged virus particles fall under the term “charge variants” if they are differently charged due to their nucleic acid (DNA or RNA) content, their glycosylation, or amidation or oxidation of the amino acids of their capsid proteins.

In the method according to the invention, specially prepared test vessels, in particular microtiter plates, are used.

If fluorescence markers are used in the method according to the invention, the PAIA plate as defined here is used.

If biosensors are used as functionalized surfaces in the method according to the invention, PAIA plates are not used.

The advantage of the invention is that a large number of samples can be tested for the presence of charge variants in a relatively short time. Due to the simple and, if desired, automated implementation, a measurement result for more than 100 samples is usually available in less than 60 minutes. The automation available for microtiter plates (e.g., pipetting robots or automatic readout methods) can be used.

Microtiter plates are well known and are usually rectangular with a varying number of wells, also known as cavities or depressions. These wells are usually arranged in rows parallel to the long side of the microtiter plate and in columns parallel to the short side.

Letters are usually used to designate the rows and numbers to designate the columns. The wells of a plate with 384 wells are arranged in 16 rows (from A to P) and 24 columns (from 1 to 24), for example. A schematic drawing of such a 384-well plate is shown in FIG. 1.

The wells of a 96-well plate are arranged in 8 rows (A to H) and 12 columns (1 to 12). This allows each well to be uniquely identified: The first row of a 96-well plate contains 12 wells, namely wells A1 to A12. The second row contains wells B1 to B12, and so on.

The special preparation of the microtiter plate involves one or more wells or, in the case of the PAIA plate, as described in more detail below, one or more wells that form a measuring chamber.

The wells of the microtiter plate are prepared as follows:

In a first step, the desired number of wells is filled with aqueous buffer solutions that have different pH values and/or different salt concentrations. These solutions contain different salt concentrations and pH values.

The pH differences between the wells can be varied depending on the requirements of the experiment (assay). This also applies to the number of wells over which the assay is spread. FIGS. 1b and 1c show a possible arrangement with a 12-point or 12-step gradient.

The intervals between the pH values are selected according to the accuracy required for the experiment. Intervals between the pH values in the individual wells in the range of 0.02 to 1 or 0.05 to 1.5 or 0.03 to 2 are advantageous. Larger intervals are also possible.

Alternatively or additionally, the buffer solution may have different salt concentrations. The intervals between the salt concentrations are selected as required by the accuracy of the experiment. It is advantageous to have intervals between the concentrations in the individual wells in the range of 0.5 to 500 mMol/L or 0.5 to 100 mMol/L or 1 to 90 mMol/L or 0.8 to 20 mMol/L or 1.5 to 50 mMol/L or 10 to 75 mMol/L. Salts that are suitable for producing such salt gradients in buffers are usually alkalin and alkaline earth metal salts such as NaCl, KCl, MgCl₂, CaCl₂), MgSO₄ mixtures or combinations thereof. All suitable salts can be used.

If the buffer solutions contain different salt concentrations and pH values, this is a combination of pH and salt gradients. This combination is referred to in the literature as a “salt-mediated pH gradient” and is used to optimize the separation performance of chromatography.

Goyon et al. (2020) describe various mixing regimes consisting of three buffers, starting from a 20 mM MES buffer (2-(N-morpholino) ethanesulfonic acid) with pH 5.6 (buffer A), a buffer consisting of 20 mM MES and 20 mM potassium HEPES (2-[4-(2-hydroxyethyl) piperazin-1-yl]ethan-1-sulfonic acid) with pH 6.8 (buffer B) and a 20 mM HEPES buffer with pH 8.2 and 100 mM potassium chloride as the salt component.

If, for example, charge variants with very different charges are to be distinguished in a mixture, it is important to cover a large pH range with the gradient in order to detect all charge variants. In this case, it is advantageous if the distance between the pH values (pH steps) is approximately 0.3 to 1.5 pH units. These gradations can, but do not have to, be the same from step to step. For such tests, at least eight different values are usually required (so-called 8-step gradient).

If the task requires the differentiation of different charge variants with only slight charge differences in a mixture, the use of smaller pH differences between the steps of the pH gradient, e.g., 0.02 to 0.1 pH units, is advantageous.

It is also advantageous to increase the number of wells for the gradient in order to cover a sufficiently large pH range at the same time. The same applies to concentration differences when forming a salt gradient.

For the concentration differences when forming a salt gradient, the above statements on the distinction between charge variants with large or small charge differences apply accordingly, i.e., to improve the resolution in a salt gradient, the concentration differences between the individual wells in the salt gradient must be small.

The above applies analogously when salt and pH gradients are used in the method according to the invention.

The water can now be removed from the wells by drying, preferably at an elevated temperature, i.e., a temperature greater than 18° C., at normal pressure or under reduced pressure.

This has the advantage that the microtiter plate prepared in this way can be stored. Of course, the assignment of the wells to the different pH values and/or salt concentrations must be noted. This can be done separately or on the plate.

For the experiment, it has proven advantageous to add the buffers with different pH values and/or salt concentrations to wells next to or below each other (see FIG. 1) so that rows or columns with increasing or decreasing pH values and/or salt concentrations are created. This makes it easier to evaluate the results.

The buffers with different pH values and/or salt concentrations are most easily obtained by mixing two buffers with different pH values and/or salt concentrations. If a buffer A with a low pH value and/or salt concentration and a buffer B with a high pH value and/or salt concentration are available, the desired pH value or salt concentration can be set by selecting a suitable mixing ratio.

Buffers with different pH values and salt concentrations can be produced by mixing two buffers, a buffer A and a buffer B. Both buffers have different pH values and one of the buffers additionally contains a specific salt concentration.

All common and known chemicals can be used as buffer substances, and the choice depends on the application of the assay. Common buffer systems used in handling proteins are, for example, PBS systems (phosphate-based saline buffer), ammonium acetate or citrate-based buffers.

A suitable buffer system is the CX-1 buffer system from Thermo Fisher Scientific, which provides a very linear buffer gradient. It consists of two buffers with pH values of 5.6 and 10.2, each containing four zwitterionic buffer substances. The desired pH values are set by mixing these two buffers in different volume ratios.

A 12-step buffer system with narrow pH intervals between buffers P1 to P12, consisting of the buffer substances Na₂HPO₄ (buffer A) and NaH₂PO₄ (buffer B) in a concentration of 0.02 M each, can be obtained by mixing buffers with pH 5.7 and pH 8.1 as follows.

For example: P1 is the buffer with pH 5.8, P2 is buffer 2 with pH 6.0, etc.

TABLE 1
Example of a 12-step buffer system
made by using two PBS buffers

| | Buffer A pH = | Buffer B pH = | Buffer A/B |
|---|---|---|---|
| No. | 5.7 [mL] | 8.1 [mL)] | mixture pH value |
| P1 | 4 | 46 | 5.8 |
| P2 | 6.15 | 43.85 | 6.0 |
| P3 | 9.25 | 40.75 | 6.2 |
| P4 | 13.25 | 36.75 | 6.4 |
| P5 | 18.75 | 31.25 | 6.6 |
| P6 | 24.5 | 25.5 | 6.8 |
| P7 | 30.5 | 19.5 | 7.0 |
| P8 | 36.0 | 14.0 | 7.2 |
| P9 | 40.5 | 9.5 | 7.4 |
| P10 | 43.5 | 6.5 | 7.6 |
| P11 | 45.75 | 4.25 | 7.8 |
| P12 | 47.35 | 2.65 | 8.0 |

The production of such multi-step (quasi-linear) buffer gradients is well known and there are now configurators available on the Internet (see, for example, https://www.aatbio.com/resources/buffer-preparations-and-recipes/phosphate-buffer-ph-5-8-to-7-4).

To produce salt gradients, i.e., buffers with different salt concentrations, buffer systems with salts dissolved in different concentrations are also and mixed. The pH of these solutions is the same.

In a special embodiment, the invention relates to a method for analyzing charge variants of proteins using specially prepared PAIA plates (i.e., in whose wells there are different pH values and/or salt concentrations, as described above) using anion or cation exchangers, mixed-mode or multi-mode ion exchangers as functionalized surfaces, and fluorescent markers as markers.

The PAIA plate is a microtiter plate in SBS format with a specific number of wells that are designed as measuring chambers in which the elevation (referred to as a structural element in WO 2015/135840 A1) is shaped so that its base area occupies at least 50% of the bottom area of a measuring chamber or well. They are known and described in WO 2015/135840 A1. They are manufactured and distributed under the trade name PAIAplate 384 by PAIA Biotech GmbH. These plates have 384 wells.

It goes without saying that the PAIA plate to be used may have more or less than 384 wells.

Preferably, the elevation has a square base that tapers (at least slightly) toward the top and has the shape of a four-sided pyramid. The upper end of the elevation is pointed, flat, or convex, preferably tapering to a point. Advantageously, the upper end of the elevation has a diameter of less than 50 μm so that no test components can deposit there. The height of the elevation is at least 10% and at most 50% of the height of the measuring chamber or well, preferably at least 15% and at most 30%. The bottom of the measuring chamber or well is opaque except for the bottom of the elevation. The elevation, on the other hand, is transparent and forms a measuring window that is used to measure the fluorescence emission, as described in detail in WO 2015/135840 A1.

The PAIA plate is equipped in particular as follows: The desired number of wells (some or all) of the microtiter plate are designed as measuring chambers, in which there is a raised area that is shaped so that its base area occupies at least 50% of the bottom area of a measuring chamber or well and preferably has a square base area, which tapers at least slightly towards the top and has the shape of a four-sided pyramid, the upper end of the elevation being pointed, flat or convex and, advantageously, the upper end of the elevation having a diameter of less than 50 μm, so that no test components can be deposited there, wherein the height of the elevation is at least 10% and at most 50% of the height of the measuring chamber or well, and the bottom of the measuring chamber or well is opaque except for the bottom of the elevation, and the elevation is transparent and forms a measuring window which is used to measure the fluorescence emission.

With regard to the measuring windows, reference is made to the description in WO 2015/132840 A1, which is incorporated herein in its entirety. For fluorescence measurement, the measuring chamber or microtiter plate according to the invention is irradiated from below with excitation light, which is then irradiated into the sample via the elevation. Commercially available fluorescence readers can be used to read the fluorescence signal.

The following devices, for example, can be used as fluorescence readers in accordance with the invention: The devices of the Spectramax series from Molecular Devices, the systems from Tecan (Safire, the Infinite series, or SPARK), readers from BMG Labtech (Omega, Clariostar, Pherastar), and fluorescence microscopes, e.g., from SynenTec (Cellavista and NyONE).

The invention using the PAIA plate can be used advantageously in particular in the development of cell culture processes for the production of proteins, in particular therapeutically active proteins such as antibodies, Fc fusion proteins, antibody fragments, bi- and multi-specific antibodies, and virus particle-based therapeutics. The invention therefore also relates to the use of PAIA plates for analysis, i.e., detection and verification of charge variants of proteins, in particular therapeutically active proteins, such as antibodies, Fc fusion proteins, antibody fragments, bi- and multi-specific antibodies, and virus particle-based therapeutics.

In one embodiment of the invention, the method according to the invention uses a microtiter plate in whose wells buffer substances and/or salts are present which, in dissolved state, result in different pH values and/or different salt contents. Such plates are produced by introducing buffer solutions and/or salt solutions with different pH values and/or salt contents into the wells of the plates and drying them at elevated temperatures, advantageously at temperatures of 18° C. or above, preferably 25° C. or above, in particular 30° C., at normal pressure or reduced pressure.

The invention also relates to a PAIA plate in which buffer solutions with different pH values and/or salt concentrations are present in one or more of the wells and in which functionalized surfaces, anion or cation exchangers, mixed-mode or multi-mode ion exchangers and, if desired, fluorescent markers are introduced.

The method according to the invention using the PAIA plate can be carried out as follows:
(a) addition of one aliquot of the protein sample to each of the wells pretreated with buffers with different pH values and/or salt concentrations.
(b) addition of anion or cation exchangers, mixed-mode or multi-mode ion exchangers that bind to the protein in a charge-specific manner, in the form of particles;
(c) addition of one or more fluorescent markers selected so that they bind specifically to the protein to be determined, whose charge variants are to be determined;
(d) mixing;
(e) measuring the intensity of the unbound fluorescent markers after settling of the anion or cation exchangers, mixed-mode or multi-mode ion exchangers;
(f) correlating the measurement with the pH values and/or salt contents present in the wells to determine the different charge variants of the protein.

In the above method, steps (a), (b) and (c) can be carried out in any order. If step (b) and/or (c)—in any order—are carried out before step (a), a drying step may be provided after step (b) and/or step (c). In the drying step, the liquid introduced with the test components is removed, preferably by drying at an elevated temperature, as described here.

If a drying step is inserted, the process can be interrupted for any length of time after the drying step and continued with step (a).

The invention relates to the PAIA plate in which, after the buffer solutions, and if necessary the functionalized surfaces and/or fluorescent markers, have been introduced, the liquid components (water from the buffer solution, etc.) are removed by drying.

The invention therefore also relates to a PAIA plate with dried substances, namely buffer substances, optionally functionalized surfaces and/or optionally fluorescent markers, wherein in some of the wells arranged in columns or rows, there is a decreasing or increasing pH value and/or salt concentration upon addition of a sample solution.

The invention also relates to a so-called kit of parts comprising a PAIA plate with dried substances, namely buffer substances and functionalized surfaces, and a fluorescent marker, wherein in some of the wells arranged in columns or rows, there is a decreasing or increasing pH value and/or salt concentration upon addition of a sample solution.

The invention can also be carried out using biosensors as functionalized surfaces in microtiter plates. The use of a PAIA plate is then not intended.

Therefore, the invention also relates to a method for analyzing charge variants of proteins using microtiter plates whose wells have at least partially different pH values and/or salt concentrations and using biosensors as functionalized surfaces. The biosensors are equipped so that they are capable of acting as anion and cation exchangers or as mixed-mode or multi-mode ion exchangers. The method may also comprise the addition of marker molecules.

The method then comprises the following steps:
(a) use of the specially pretreated microtiter plate, in whose wells different pH values and/or salt concentrations are present, whereby not every well of the plate needs to be occupied, and
(b) adding one aliquot of the protein sample to each of the wells pretreated with buffers of different pH values and/or salt concentrations;
(c) insertion of the biosensor into the treated wells of the microtiter plate containing the sample;
(d) mixing;
(e) measuring the signal triggered by the biosensor;
(f) correlating the measurement with the pH value and/or salt content present in the wells to determine the different charge variants of the protein.

The method according to the invention for analyzing charge variants of proteins using biosensors can also be carried out as follows:
(1) using the specially prepared microtiter plate in whose wells a suitable pH and/or salt gradient is present, wherein the pH or salt gradient is present in dried form or is freshly applied;
(2) adding one aliquot of the protein sample to each of the wells pretreated with buffers of different pH values and/or salt concentrations;
(3) inserting the biosensors with ion exchange surfaces into the cavities with the pH and/or salt gradient;
(4) shaking the microtiter plate, thereby accelerating the reaction, and simultaneously (in real time) measuring the binding to the ion exchange surfaces on the biosensors;
(5) if unpurified samples containing foreign proteins are used, or if the measurement signal of the target molecule is too low, shaking is stopped after the maximum measurement signal is reached (i.e., biosensors are saturated);
(6) the biosensors are removed from the microtiter plate and placed in a second microtiter plate containing the same gradient buffer and, in addition, a marker molecule that binds specifically to the target molecule;
(7) shaking of the microtiter plate again and simultaneously measuring of the binding of the marker molecule to the target proteins already bound to the ion exchange surfaces of the biosensors;
(8) determination of the binding of the target protein to the ion exchange surfaces in all wells of the gradient either via the signal measured in step (4) or in step (7) and determination of the charge profile of the sample.

The detection of charge variants using biosensors can be performed by applying ion exchange surfaces to biosensors, which are placed in the wells of microtiter plates (not PAIA plates) with different pH values or salt concentrations and where the binding of the target proteins is measured.

Optical methods such as biolayer interferometry are primarily used for this purpose, in which a phase shift of the light occurs when molecules bind to the surface of the biosensor. This phase shift is measurable and depends on the number and mass of the molecules bound to the surface.

Measurements with biosensors can be performed simultaneously, i.e., with many biosensors. Alternatively, they can be performed sequentially, i.e., a biosensor is inserted one after the other into the various cavities of the microtiter plate containing one step of the gradient and regenerated and washed before each measurement of a new well.

The regeneration of biosensors is a well-known procedure. Modern devices are capable of measuring with several biosensors simultaneously in different wells, thereby achieving a significantly higher sample throughput.

If only small amounts of a protein are bound during the measurement or very small proteins are measured and therefore only a very weak measurement signal is generated, it is common practice with biosensors to bind a marker that binds specifically to the target molecule, e.g., an antibody with a sufficiently large molecular mass, to the surface after the target molecule has bound to the surface in order to amplify the signal. If this is not sufficient to amplify the signal, another marker (here called a binder, e.g., a secondary antibody) that binds specifically to the primary antibody can also be bound to the biosensor.

The same approach is used if other unwanted molecules and proteins (“non-target molecules”) bind to the surface during protein binding. This can be the case, for example, if the sample has not been purified and contains foreign proteins that also bind to the surface.

In this case, the use of a specific marker molecule, e.g., an antibody that binds specifically to the target protein, allows the measurement of a phase shift due to the binding of the marker molecule, and this binding depends only on the amount of target molecules bound to the surface. It is therefore a measurement signal specific to the target molecule.

After collection, the sample containing the protein with the charge variants is usually added in portions to the wells of the microtiter plate, which may also be a PAIA plate, without further treatment (e.g., purification). However, it is advantageous to dilute the sample with water in order to reduce the concentration of buffer substances that could interfere with the experiment.

The portioning is done manually or automatically with an automated pipetting robot (liquid handler).

When using the PAIA plate, it may be advantageous for efficiency if the fluorescent marker(s) and/or the functionalized surfaces are already present in a dried stage in the PAIA plate.

The special feature of the invention is that, when the method is carried out in the cavities of the microtiter plate arranged in rows or columns, different pH values and/or salt concentrations are present.

A particular advantage of the invention is that the samples can be measured directly (optionally, after dilution with water). Purification prior to performing the methods (the assays) is not necessary.

The pH value and/or salt concentration present in the wells are decisive for the binding properties of the respective charge variant of the protein to the functionalized surface(s). The measurement with the biosensor or, when using the PAIA plate, the fluorescence intensity of the unbound fluorescent markers provides a value from which the charge state of the measured protein can be determined.

In the method according to the invention, the functionalized surfaces are selected so that they can bind the protein with the charge variants. Anion or cation exchangers, mixed-mode or multi-mode ion exchangers, and biosensors whose measuring surfaces have the same or similar functionalities as the aforementioned anion or cation exchangers, mixed-mode or multi-mode ion exchangers are suitable for use according to the invention.

Anion exchangers are known and are classified according to their ion strength as strong and weak anion exchangers. Strong anion exchangers are those with positively charged functional groups, e.g., quaternary aminoethyl groups, quaternary aminomethyl groups, or triethylaminomethyl groups. Weak anion exchangers often contain diethylaminoethyl groups.

Strong anion exchangers include Macro-Prep High Q Support and UNOsphere Q Media, both available from Bio-Rad, or QAE Sephadex A-25 (Cytiva), Capto Q (Cytiva), and POROS XQ Strong Anion Exchange Resin (ThermoFisher).

Weak anion exchangers include DEAE Sepharose Fast Flow (Cytiva), Macro-Prep DEAE Resin (Bio-Rad), TOYOPEARL DEAE-650 (Tosoh), DEAE Ceramic HyperD (Sartorius), and Fractogel® EMD DEAE (Merck).

Cation exchangers are equally well known and are also divided into strong and weak cation exchangers. The former contain, for example, sulfonate groups(S), sulfoethyl groups (SE) or sulfopropyl groups (SP) on their surface. Weak cation exchangers contain negatively charged groups such as carboxymethyl groups (CM).

Strong cation exchangers include SP Sepharose Fast Flow (Cytiva), SP Sephadex C-25 (Cytiva), TOYOPEARL SP-650 (Tosoh), and Macro-Prep High S (Bio-Rad).

Weak cation exchangers are well known and commercially available, such as Macro-Prep CM (Bio-Rad), CM Sepharose High Performance FF (Cytiva), G-Sep™ CM Agarose Fast Flow (G-Bioscience).

Mixed-mode and multi-mode ion exchangers are well known and commercially available.

Mixed-mode ion exchangers include the CHT (ceramic hydroxyapatite) column material types I and II from Bio-Rad, on the surface of which negatively charged phosphate groups and positively charged calcium ions are available for interaction.

Another mixed-mode or multi-mode (cation) exchanger is the material TOYOPEARL® MX-Trp-650M from Tosoh Bioscience, whose surface contains negatively charged carboxyl groups as well as hydrophobic tryptophan groups for hydrophobic interactions. Another mixed-mode ion exchanger is a weak cation exchanger with hydrophobic groups, which is available as Capto MMC column material from Cytiva. Another example is Capto Adhere (also from Cytiva), a strong anion exchanger with additional hydrophobic aromatic groups.

Preferably, anion or cation exchangers, mixed-mode or multi-mode ion exchangers that can be used according to the invention have average diameters in the range of about 5 to 200 μm. Preferably, the particles have average diameters in the range of 5 to 150 μm or 10 to 100 μm.

### DESCRIPTION OF THE FIGURES

FIG. 1a shows a schematic labeling of the wells present in a 384-well microtiter plate.

FIG. 1b shows a schematic representation of a 384-well plate with a 12-step pH gradient. The 12-step gradient is present in each column. Untreated wells are present in columns 5, 10, 14, 18, 22-24.

FIG. 1c shows a schematic representation of a 384-well plate with a 12-step pH gradient. There are two 12-step gradients per row (columns 1-12 and columns 13-24). There are no untreated wells.

FIG. 2 shows the documentation scheme from Example 1.

FIG. 3 refers to Example 1 and shows the measured fluorescence intensities of the unbound fluorescent markers of V1-A trastumab (HER)-unstressed and V1-B trastumab (HER)-heat-treated as a function of pH.

FIG. 4 refers to Example 1 and shows the fluorescence intensities of V1-C trastumab (HER)-unstressed and V1-D trastumab (HER)-heat-treated, measured in diluted cell culture supernatant.

FIG. 5a refers to Example 1 and shows a comparative example. The result of the chromatographic analysis is shown, namely a CEX chromatogram of the unstressed trastuzumab sample 2 mg/ml in H₂O.

FIG. 5b refers to Example 1 and represents a comparative example. The result of the chromatographic evaluation is shown, namely a CEX chromatogram of the trastuzumab sample 2 mg/mL in H₂O stressed at 35 degrees.

FIG. 6 refers to Example 2 and shows the course of the fluorescence intensities as a function of the pH values shown on the x-axis.

FIG. 7 refers to Example 2 and shows a representation of the point-to-point slope as a function of the pH value.

### LABELING OF FIGURES

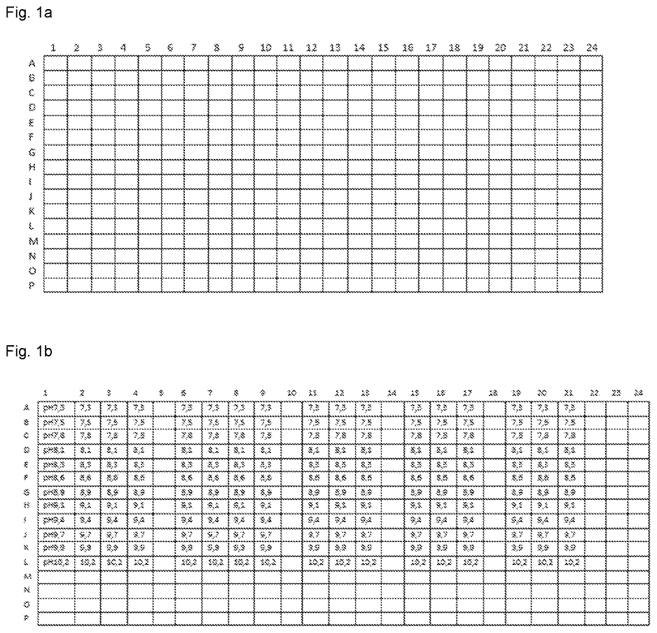

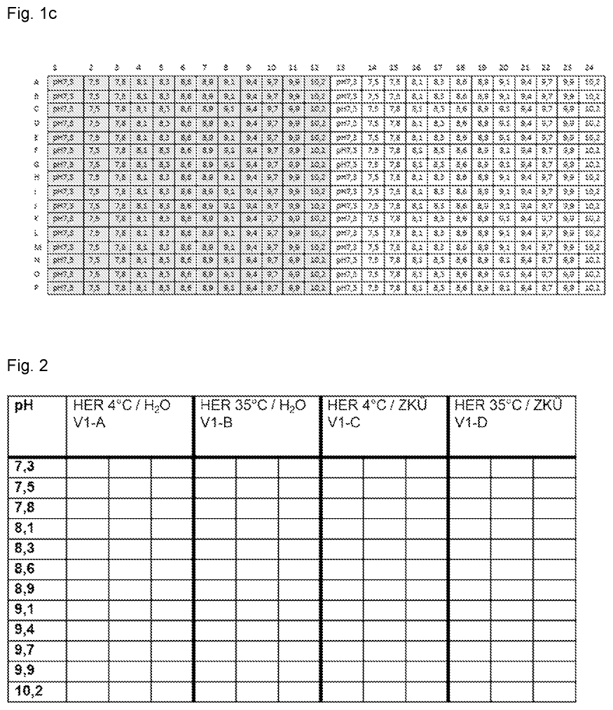

FIG. 1a: Schematic labeling of the wells present in a 384-well plate.

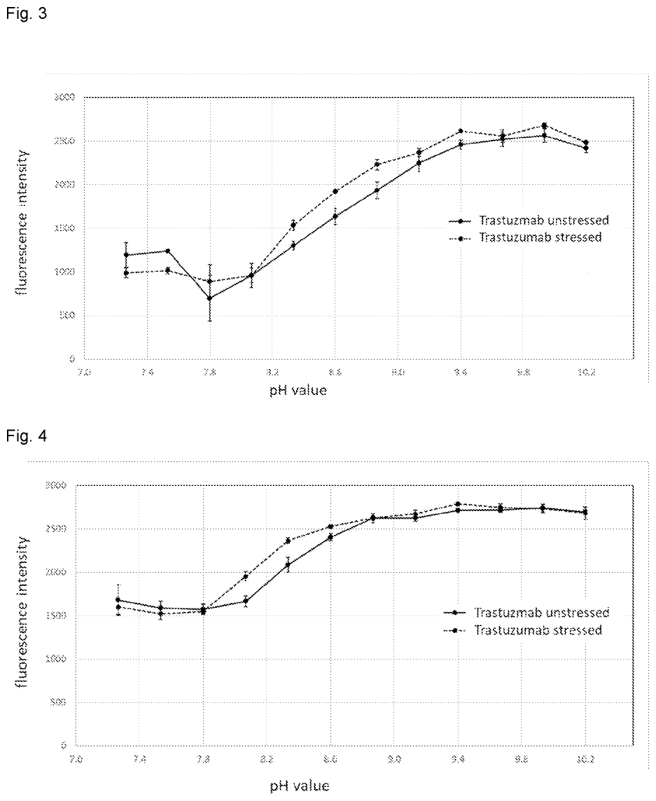

FIG. 1b: Schematic representation of a 384-well plate with a 12-step pH gradient. FIG. 1c: Schematic representation of a 384-well plate with a 12-step pH gradient. FIG. 2: Labeling scheme from Example 1.

FIG. 3: Example 1: Fluorescence intensities of the samples-samples measured in H₂O

FIG. 4: Example 1: Fluorescence intensities of the samples-samples measured in diluted cell culture supernatant

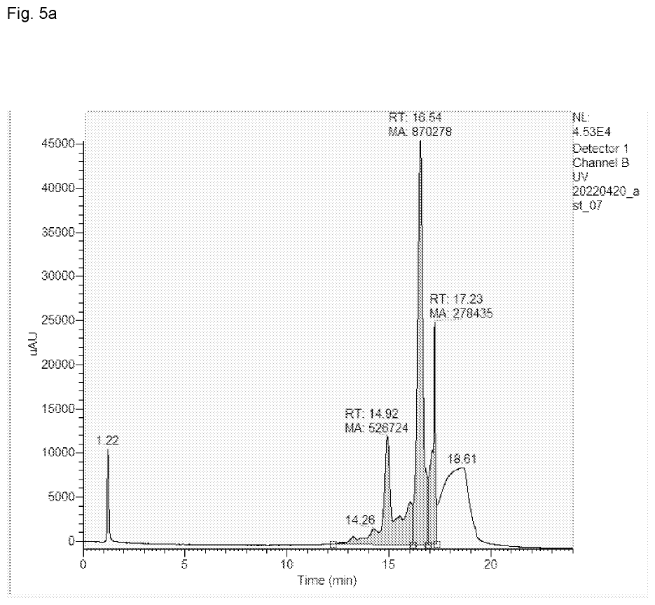

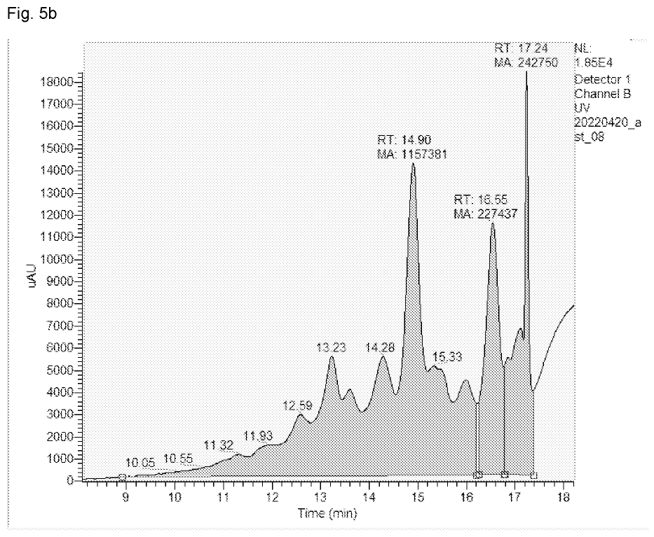

FIG. 5a: Example 1-Comparative example: Chromatographic separation of unstressed samples in H₂O

FIG. 5b: Example 1-Comparative example: Chromatographic separation of stressed samples in H₂O

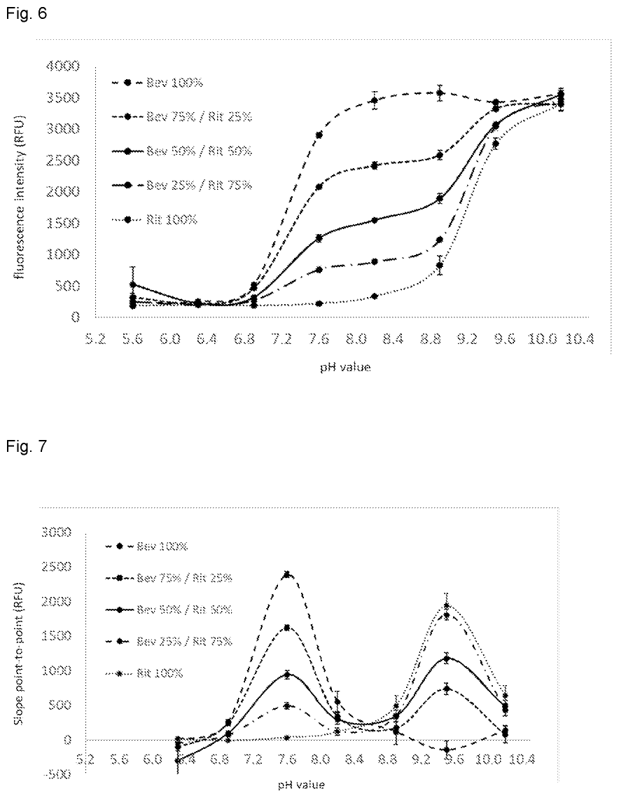

FIG. 6: Example 2-Course of fluorescence intensities as a function of pH

FIG. 7: Example 2-Representation of the point-to-point slope as a function of the pH value

The invention is explained using the examples shown in the figures and described below, without limiting the invention to these examples.

### EXAMPLES

Unless otherwise specified in the following examples 1 and 2, the following aids and reagents are used:

The wells of a PAIAplate 384 are used as reaction and measuring chambers. It is available from PAIA Biotech GmbH and is manufactured and used as described in WO 2015/135840 A1. The working volume of a well is 15 μL-110 μL.

Unless otherwise stated, a phosphate-buffered salt solution, known as PBS buffer, is used, which is adjusted to a pH value of 7.4. The PBS buffer contains 137 mM NaCl, 2.7 mM KCl, and 12 mM phosphate.

A strong cation exchanger in the form of particles, namely CaptoSP ImpRes particles (Cytiva, item no. 17546802) with an average diameter of approx. 10 μm, is used as the functionalized surface. The particles carry ligands with sulfonic acid groups.

Unless otherwise specified, the measuring chambers are treated as follows before measurement:

### Preparation of the Measuring Chambers/Microtiter Plate

A 9% particle working solution with a concentration of 10% [v/v] LPS is prepared from the cation exchange particle stock solution (slurry) by adding water and LPS (Liquid Plater Sealer, Candor).

Pipette 30 μL of this particle working solution into the corresponding wells of the PAIA plate. Add 7.5 μL of a buffer solution with different pH values to each of these wells (see the tables of examples 1 and 2 for more detailed information on the pH values), adding the buffer according to a specific pattern so that it is possible to trace which buffer pH was added to which well.

After adding the buffers, mix briefly. Then the liquid is removed from the plate. To do this, the plate is dried at 35° C. for 48 hours in a drying cabinet or similar. After drying, the plate can be stored or used directly for measurement.

### Example 1: Measurement of Charge Variants (CVAs) of Thermally Stressed Antibody Samples

### Preparation of the Buffer Gradient:

A CX-1 pH gradient system from Thermo Fisher Scientific (item no. 302779) is used. This system consists of four different buffer substances and is characterized by the fact that defined pH values can be set by mixing two buffers (buffer A and buffer B), resulting in a pH gradient with intervals between the pH values of 0.2-0.3.

Buffer A has a pH value of 5.6 and buffer B has a pH value of 10.2.

The following mixtures are used to produce a 12-point gradient in the range from 7.3 to 10.2.

TABLE 2
12-point gradient, produced with CX-1 buffer
system from Thermo Fisher Scientific

| | Buffer A pH = | Buffer B pH = | Buffer A/B |
|---|---|---|---|
| No. | 5.6 [% (v/v)] | 10.2 [% (v/v)] | mixture pH value |
| P | 64 | 36 | 7 |
| P2 | 58 | 42 | 7.5 |
| P3 | 52 | 48 | 7.8 |
| P4 | 46 | 54 | 8.1 |
| P5 | 41 | 59 | 8.3 |
| P6 | 35 | 65 | 8.6 |
| P7 | 29 | 71 | 8.9 |
| P8 | 23 | 77 | 9.1 |
| P9 | 17 | 83 | 9.4 |
| P10 | 12 | 88 | 9.7 |
| P11 | 6 | 94 | 9.9 |
| P12 | 0 | 10 | 10.2 |

The measuring chambers in the PAIA plate are prepared as described above, whereby 7.5 μL of buffer solutions P1 to P12 are added to each of the wells containing particles, as shown in Table 2. The buffers are added according to a specific pattern so that it is possible to trace which of the buffers P1 to P12 is present in which well. Alternatively, it can be noted in which well the respective buffer P1 to P12 is present.

The following samples were tested, with the target protein being the antibody trastuzumab:
V1-A: Trastuzumab (HER, Herceptin) stored at 4° C.; sample diluted with water before measurement.
V1-B: Trastuzumab stored at 35° C. for 14 days; sample diluted with water before measurement.
V1-C: Trastuzumab (HER, Herceptin) stored at 4° C.; sample diluted with cell culture supernatant (CCS) before measurement.
V1-D: Trastuzumab (HER, Herceptin) stored at 35° C. for 14 days; sample diluted with cell culture supernatant (CCS) before measurement.

Heat treatment produces acidic charge variants of trastuzumab.

The trastuzumab samples are diluted with water or cell culture supernatant diluted with water (dilution 1:5) to a concentration of 150 μg/mL.

10 μL of this sample solution were added to 3 of the wells treated with P1 to P12. This means that 36 wells are occupied and three data values are obtained for each buffer P1 to P12. Add 50 μL of the fluorescent marker (with Alexa 647-conjugated Affibody (Affibody AB, Sweden)) to these occupied wells and mix everything.

Add 50 μL of the above-mentioned fluorescent marker, which is present in a concentration of 12 nM in a PBS buffer with 50 ppm BSA.

The plate layout is shown in FIG. 2 as an example.

The entire microtiter plate was shaken at room temperature at 1400 rpm on an Eppendorf Thermomixer Comfort for 30 minutes and then centrifuged at 500×g for one minute.

The fluorescence intensity is then measured from below (bottom reading) in each measurement chamber in a Tecan Safire fluorescence plate reader at an excitation wavelength of 630 nm and an emission wavelength of 665 nm.

FIGS. 3 and 4 illustrate the differences with regard to the charge variants present, i.e., the CVA profiles of the temperature-treated samples V1-B and V1-D compared to the non-temperature-treated samples V1-A and V1-C.

The curves of the temperature-treated and thus stressed samples show a shift in the increase in fluorescence to lower (acidic) pH values, i.e. there is a proportion of trastuzumab antibodies in these samples that are already detached from the particles (or bind more weakly to the particles) in slightly acidic buffers, suggesting that these antibodies are acidic charge variants of trastuzumab.

To verify the results, the same samples are analyzed using conventional cation exchange chromatography. The system uses a Column Thermo ProPac WCX-10, 250×4 mm cation exchange column and the CX-1 buffer system from ThermoFisher with a flow rate of 1 mL/min. The starting buffer consists of 100% buffer A, to which buffer B is gradually added during the first 15 minutes until a concentration of 50% B is reached. In the following 2 minutes, the proportion of buffer B is increased to 100%. This results in a pH gradient buffer of 5.6 to 10.2. The eluted molecules are detected by absorption measurement at 280 nm. The results are shown in FIGS. 5a and 5b.

The results of the experiments according to the invention and using the conventional method (CEX chromatography) agree well. The measurements of the samples in water and the diluted cell culture supernatant also agree.

Looking at the results of conventional cation exchange chromatography, it is noticeable that the main peak, i.e., the charge variant most frequently occurring in the unstressed trastuzumab sample, is eluted from the column after a retention time of 16.54 minutes. The peak area is 870,278 AU.

Before this main peak, an increase in the baselines can be seen, indicating the presence of some acidic charge variants, as well as an acidic peak at a retention time of 14.92 minutes. Overall, the acidic variants have a peak area of 526,724 AU.

A basic charge variant is also recorded in this sample at a retention time of 17.23 minutes with a peak area of 278,435 AU.

In the sample stressed at 35° C., the main peak (retention time 16.55 minutes) is significantly smaller than in the unstressed sample and has a significantly smaller peak area (227,437 AU).

The acidic charge variants are significantly more present in the stressed sample and have a cumulative peak area of 1,157,381 AU.

The basic peak detected in both samples has a virtually identical peak area in both chromatograms.

This means that the temperature treatment generates acidic charge variants. This is also described in the literature (Spanov et al. Change of charge variant composition of trastuzumab upon stressing at physiological conditions, Journal of Chromatography A 1655 (2021) 462506).

### Example 2: Determination of Two Antibodies with Different Isoelectric Points

The buffer solutions with different pH values are prepared as described in Example 1, using the 8-step buffer system according to Table 3, as shown below.

TABLE 3
8-point gradient, produced with CX-1 buffer
system from Thermo Fisher Scientific

| | Buffer A pH = | Buffer B pH = | Buffer A/B |
|---|---|---|---|
| No. | 5.6 [% (v/v)] | 10.2 [% (v/v)] | mixture pH |
| P1 | 10 | 0 | 5.6 |
| P2 | 85 | 15 | 6.3 |
| P3 | 72 | 28 | 6.9 |
| P4 | 57 | 43 | 7.6 |
| P5 | 43 | 57 | 8.2 |
| P6 | 28 | 72 | 8.9 |
| P7 | 15 | 85 | 9.5 |
| P8 | 0 | 100 | 10.2 |

The fluorescent marker used is Nano-Secondary® alpaca anti-human IgG/anti-rabbit IgG, recombinant VHH, Alexa Fluor® 647 (item no. CTK0101-ChromoTek GmbH). This is used at a concentration of 5 nM in a PBS buffer with 50 ppm BSA.

The biological analytes used are the two monoclonal antibodies bevacizumab (Bev) and rituximab (Rit) as well as three mixtures of these two molecules in mixing ratios of 75:25, 50:50, and 25:75. The total concentration of antibodies in all samples was 50 μg/mL.

10 μl of these sample solutions are added to three wells with different pH values (a total of 24 cavities per sample) and mixed with 50 μl of the fluorescent marker solution. The entire microtiter plate is shaken at room temperature for 30 minutes at 1400 rpm in an Eppendorf Thermomixer Comfort and then centrifuged for one minute at 500×g.

The fluorescence intensity is then measured from below (bottom reading) in each measuring chamber in a Tecan Safire fluorescence plate reader at an excitation wavelength of 630 nm and an emission wavelength of 665 nm.

The diagrams in FIG. 6 show the fluorescence intensity curves of the different samples as a function of the pH value in the wells.

The sample with 100% Bev shows the greatest difference in fluorescence intensity between pH values 6.9 and 7.6. As the Bev content in the samples decreases, this difference decreases and, at the same time, the increasing Rit content in these samples causes the fluorescence intensity in these samples to increase sharply between pH values of 8.9 and 9.5.

This means that Bev is already separated from the particles at pH values of approx. 7, while Rit is only separated at pH values of approx. 9. This means that Bev is a significantly more acidic antibody than Rit. This corresponds to the isoelectric points of 8.5 for Bev and 9.5 for Rit specified in the literature. (See Goyon et al. 2017: Determination of isoelectric points and relative charge variants of 23 therapeutic monoclonal antibodies Journal of Chromatography B Volumes 1065-1066, 15 Oct. 2017, pp 119-128).

If the point-to-point slope of the samples is plotted instead of the fluorescence intensity, a chromatogram-like representation is obtained in which peaks are visible (FIG. 7). The height of the peaks also corresponds to the mixing ratios set in the samples, i.e., quantitative statements can be made.

1. A method for analyzing charge variants of proteins using microtiter plates whose wells have at least partially different pH values and/or salt concentrations and using functionalized surfaces.

2. The m method according to claim 1, wherein the proteins are therapeutically active proteins, such as antibodies, Fc fusion proteins, antibody fragments, bi- and multi-specific antibodies, and virus particle-based therapeutics.

3. The m method according to claim 1, wherein the charge variants comprise mismatched and misassembled proteins and their fragments that occur during production or virus particle-based therapeutics that have a defective nucleic acid loading.

4. The m method according to claim 1, wherein the proteins are virus particles that can be loaded with nucleic acids.

5. The m method according to claim 1, wherein the wells of the microtiter plate are prepared as follows:
filling the wells in the microtiter plate with aqueous buffer solutions having different pH values, wherein the pH values in the individual wells are in the range of 0.02 to 2, wherein not all wells need to be filled; and/or
filling the wells in the microtiter plate with aqueous buffer solutions having different salt concentrations, wherein the intervals between the concentrations in the individual wells are in the range from 0.5 to 100 mMol/L, wherein not all wells need to be filled; or
filling the wells in the microtiter plate with aqueous buffer solutions having different pH values and salt concentrations, wherein the pH values in the individual wells are spaced apart in the range from 0.02 to 2 and the salt concentrations are spaced apart in the range from 0.5 to 100 mMol/L, wherein not all wells need to be filled; and
optionally removing the water from the wells.

6. The m method according to claim 5, wherein the buffers with different pH values and/or salt concentrations are introduced into wells located next to or below each other so that rows or columns with ascending or descending pH values and/or salt contents are formed.

7. The m method according to claim 1, using marker molecules that bind specifically to the protein whose charge variants are being analyzed.

8. The m method according to claim 7, wherein the marker molecules are monoclonal or polyclonal antibodies or nanobodies or single-domain antibodies derived therefrom, as well as peptides and RNA- or DNA-based aptamers, and these form fluorescent markers, if necessary coupled to fluorescent dyes.

9. The m method according to claim 7, wherein
the functionalized surfaces are anion or cation exchangers,
mixed-mode or multi-mode ion exchangers,
the marker molecules are fluorescent markers, and
the microtiter plate is equipped as follows: some or all of the wells of the microtiter plate are designed as measuring chambers; in which a elevation is located, which is shaped such that its base area occupies at least 50% of the bottom area of a measuring chamber or well and preferably has a square base area that tapers at least slightly toward the top and has the shape of a four-sided pyramid, wherein the upper end of the elevation is pointed, flat or convex, and advantageously the upper end of the elevation has a diameter of less than 50 μm, so that no test components are deposited there, wherein the height of the elevation is at least 10% and at most 50% of the height of the measuring chamber or the well, and the bottom of the measuring chamber or the well is opaque except for the bottom of the elevation, and the elevation is transparent and forms a measuring window which is used to measure the fluorescence emission.

10. The m method according to claim 9, comprising the following steps:
a. using the microtiter plate, the wells of which have been at least partially pretreated by:
filling the wells in the microtiter plate with aqueous buffer solutions having different pH values, wherein the pH values in the individual wells are in the range of 0.02 to 2, wherein not all wells need to be filled; and/or
filling the wells in the microtiter plate with aqueous buffer solutions having different salt concentrations, wherein the intervals between the concentrations in the individual wells are in the range from 0.5 to 100 mMol/L, wherein not all wells need to be filled; or
filling the wells in the microtiter plate with aqueous buffer solutions having different pH values and salt concentrations, wherein the pH values in the individual wells are spaced apart in the range from 0.02 to 2 and the salt concentrations are spaced apart in the range from 0.5 to 100 mMol/L, wherein not all wells need to be filled; and
optionally removing the water from the wells;
b. adding one aliquot of the protein sample to each of the pretreated wells;
c. adding anion or cation exchanger, mixed-mode or multi-mode ion exchanger that binds charge-specific to the protein, in the form of particles;
d. adding one or more fluorescent markers which are selected so that they bind charge-selective to the protein whose charge variants are to be determined;
e. mixing;
f. after the anion or cation exchangers, mixed-mode or multi-mode ion exchangers are settled, measuring the intensity of the unbound fluorescent markers;
g. correlating the measurement with the pH values and/or salt contents present in the wells to determine the different charge variants of the protein.

11. The m method according to claim 1, wherein
the functionalized surfaces are biosensors capable of acting as anion and cation exchangers or as mixed-mode or multi-mode ion exchangers; and
wherein, optionally marker molecules are present, in particular monoclonal or polyclonal antibodies or nanobodies derived therefrom or single-domain antibodies, as well as peptides and RNA- or DNA-based aptamers.

12. The m method according to claim 11, comprising the following steps:
a. using the microtiter plate whose wells have been at least partially pretreated by:
filling the wells in the microtiter plate with aqueous buffer solutions having different pH values, wherein the pH values in the individual wells are in the range of 0.02 to 2, wherein not all wells need to be filled; and/or
filling the wells in the microtiter plate with aqueous buffer solutions having different salt concentrations, wherein the intervals between the concentrations in the individual wells are in the range from 0.5 to 100 mMol/L, wherein not all wells need to be filled; or
filling the wells in the microtiter plate with aqueous buffer solutions having different pH values and salt concentrations, wherein the pH values in the individual wells are spaced apart in the range from 0.02 to 2 and the salt concentrations are spaced apart in the range from 0.5 to 100 mMol/L, wherein not all wells need to be filled; and
optionally removing the water from the wells the method according to claim 5 or 6;
b. adding one aliquot of the protein sample to each of the wells pretreated with buffers having different pH values and/or salt concentrations;
c. introducing the biosensor into the treated wells of the microtiter plate containing the sample;
d. mixing;
e. measuring the signal triggered by the biosensor;
f. correlating the measurement with the pH value and/or salt content present in the wells in order to determine the different charge variants of the protein.

13. A microtiter plate produced according to one of the methods according to claim 5 in a dried state.

14. The microtiter plate according to claim 13, wherein the following process steps are carried out before drying, namely addition of anion or cation exchangers, mixed-mode or multi-mode ion exchangers in the form of particles.

15. A kit containing the microtiter plates according to claim 13 and a marker molecule.
